(19) United States
(12) Patent Application Publication
(10) Pub. No.: US 2019/0201327 A1
(43) Pub. Date: Jul. 4, 2019

(54) TOPICAL APPLICATIONS OF WITHAFERIN A

(71) Applicant: AMPERSAND BIOPHARMACEUTICALS, INC., Thousand Oaks, CA (US)

(72) Inventor: Bruce J. Sand, Thousand Oaks, CA (US)

(73) Assignee: AMPERSAND BIOPHARMACEUTICALS, INC., Thousand Oaks, CA (US)

(21) Appl. No.: 16/198,700

(22) Filed: Nov. 21, 2018

Related U.S. Application Data
(60) Provisional application No. 62/562,725, filed on Sep. 25, 2017.

(51) Int. Cl. A61K 9/00; A61K 47/14; A61K 47/10; A61K 47/12; A61K 47/02; A61K 31/196; A61K 47/24; A61K 9/06; A61K 31/585

(52) U.S. Cl. CPC A61K 9/0014; A61K 47/14; A61K 47/10; A61K 47/12; A61K 31/585; A61K 31/196; A61K 47/24; A61K 9/06; A61K 47/02

(57) ABSTRACT

This disclosure is directed to the topical/transdermal administration of the phytochemical, Withaferin A (WFA), for its wide range of pharmacological applications to include its anti-cancer, anti-obesity, anti-diabetic, anabolic effect on osteoporotic bone and its many other proteasomal inhibitory effects.

### CROSS-REFERENCE TO RELATED APPLICATIONS

This application is a non-provisional patent application and claims the benefit of U.S. provisional patent application Ser. No. 62/562,725, filed Sep. 25, 2017, and entitled “TOPICAL APPLICATIONS OF WITHAFERIN A”, whereby the contents of the aforementioned applications are incorporated herein by reference.

### BACKGROUND

The subject of this patent application relates generally to topical administration of medicinal plants, and more particularly to topical administration of Withaferin A (WFA) configured for anti-cancer properties.

Applicant(s) hereby incorporate herein by reference any and all patents and published patent applications cited or referred to in this application. By way of background, the disclosure herein relates generally to topical treatments that effect transdermal transport of carbonate salts through the skin. More particularly, it concerns direct application of a penetrating formulation containing a carbonate salt topically to a subject.

Plants have always been in the fore-front of new drug discovery. A large number of plant constituents, commonly known as phytochemicals, have gained the status of clinically used modern medicines, while many others have served as potential lead components for designing novel therapeutics. In fact, plants are natural chemical factories for synthesizing chemicals with structural diversity, such as steroids, therpenoids, flavonoides, alkaloids, etc. Many of these plant secondary metabolites are biologically active and can interact directly or indirectly with various cellular components, especially, proteins and lipids, thereby reversing the altered functions of cancer cells. Since the growth and development of tumors are triggered by oxidative stress and chronic inflammation, phytochemicals with antioxidative and anti-inflammatory properties are thought to play important roles in the prevention and/or treatment of cancer. Until now, numerous phytochemicals have been examined for their potential anticancer properties by using a diverse array of preclinical experimental models.

A large number of laboratory-based studies have demonstrated the anti-cancer effect of the medicinal plant, Withania somnifera. These anti-cancer properties are attributable to withanolides, a class of bioactive constituents isolated from Withaferin A (WFA).

WFA is a steroidal lactone, derived from Acnistus arboescens, Withania somnifera and other members of Solanaceae family. It has been traditionally used in ayurvedic medicine. It is the first member of the withanolide class of ergostane type product to be discovered. This natural product has a wide range of pharmacological activities. including anti-carcinogenic, anti-metastasis, anti-angiogenesis, cardioprotective and immune-modulatory properties. Withanolides are a group of naturally occurring C28-steroidal lactones. They contain four cycloalkane ring structures, three cvclohexane rings and one cyclopentane ring. WFA is highly reactive because of the ketone containing unsaturated A ring, and the epoxide in B ring and unsaturated lactone ring. The double bond in ring A and the epoxide ring are mainly responsible for the cytotoxicity. The 22^{nd} and the 26^{th} carbon of ergostane skeleton in WFA and related steroidal compounds are oxidized to form six membered delta lactone unit.

Systematic research on the evaluation of anticancer activities of WFA was started around the 1970s. Since then, a large number of studies have demonstrated the ability of WFA to suppress the in vivo growth of various human cancer cells' xenograph tumors, as well as experimentally induced carcinogenesis in different animal models.

In a pioneering study, Shohat reported that the administration of WFA reduced the growth of Ehrich ascites tumor. Yang et al. reported the mechanism-based anticancer activity of WFA, which reduced the growth of human prostate cancer. The in vivo growth of various human gynecological cancer cells was also attenuated by WFA. The compound inhibited the growth of human breast cancer in nude mice associated with increased apoptosis and deregulated tumor cell metabolism. WFA has been reported to suppress mouse melanoma tumor cell growth in vivo. Administration of WFA to medullary thyroid cancer cells xenograft tumor-bearing nude mice showed significant inhibition of tumor growth and metastasis. WFA also inhibited the growth of pancreatic cancer cells xenograft tumor and human colon cancer.

WFA has been shown to inhibit cancer cell motility and invasion in wound healing by selectively suppressing MMP-2. It enhances the secretion of Par-4, which in turn suppresses MMP-2 expression that is required for tumor metastasis. 3-azido Withaferin A, a stable derivative of WFA, acts as a tumor suppressor by inducing Par-4, TIMP-1 and by reducing the levels of pAkt and pERK that are activated in various cancers. These findings have augmented the therapeutic potential of the pro-apoptotic protein Par-4 in cancer. It has been demonstrated that 3-azido WFA abrogated neovascularization in vivo in a dose dependent manner.

WFA inhibits NF-kB transcription factor that regulates genes that are involved in angiogenesis. It also inhibits other transcription factors, which are important mediators of many signaling pathways that are down-regulated by various chemotherapeutic agents.

WFA inhibits tumor growth through ATP-independent inhibition of heat shock protein 90 in in vivo pancreatic models. The anti-tumor and anti-angiogenesis activity of WFA is due to the inhibition of chymotrypsin, whereas the induction of apoptosis is due to the inhibition of protein kinase C.

Other effects of WFA include:
1. Reduced growth of Ehrich ascites tumor in mouse model;
2. Reduced growth of human prostate cancer in nude mice;
3. Suppressed mouse melanoma;
4. Showed significant inhibition of tumor growth and metastasis in nude mice;
5. Suppressed DMBA-induced oral carcinogenesis in hamsters;
6. Inhibited growth of pancreatic cancer cell xenograft tumors in mice; and
7. Inhibited volume and weight of human colon cancer cell xenograft tumors in mice.
8. Intervention with tumor-specific biochemical processes is the mechanistic basis of anticancer effects of WFA; these include:
   a. Reinforcement of cellular antioxidant and/or detoxification systems;
   b. Suppression of inflammatory pathways;
   c. Selective inhibition of tumor cell proliferation and induction of apoptosis;
   d. Suppression of tumor angiogenesis;
   e. Blockade of epithelial-to-mesenchymal transition, tumor invasion, and metastasis;
   f. Alteration of tumor cell metabolism;
   g. Immunomodulation; and
   h. Eradication of cancer stem cells.

The inhibition of cell proliferation and induction of apoptosis are the prime mechanisms underlying anticancer effects of WFA.

The combination of WFA with doxorubicin has elicited synergistic antiproliferative and apoptosis-inducing effects, thus reducing the requirement of lower chemotherapeutic dose of doxorubicin-induced adverse drug reactions.

The foregoing highlights the extensive study and use of this natural product for cancer treatment or prevention. This product also has a wide range of other pharmacological activities in addition to the foregoing.

When scientists discovered the hormone leptin in the 1990s, many had thought studying its actions would lead to treatments for obesity. Leptin helps regulate hunger and some scientists have proposed that obesity is the result of resistance to the hormone's actions. An analysis was undertaken of a library of small molecules that have mRNA expression profiles similar to that of celastrol, a naturally occurring compound that was previously identified as a leptin sensitizer. WFA has been identified as a potent leptin sensitizer and has been demonstrated to reduce weight by 20 to 25% in obese mice, suggesting that it has antidiabetic effects. WFA also has beneficial effects on glucose metabolism, unlike celastrol.

WFA also acts as a proteasomal inhibitor. Proteasome inhibitors are drugs that block the action of proteasomes, cellular complexes that break down proteins. Multiple mechanisms are likely to be involved, but proteasome inhibition may prevent degradation of pro-apoptotic factors.

WFA. thereby, binds to specific catalytic ß subunit of the 20S proteasome. It exerts positive effect on osteoblast survival by increasing osteoblast proliferation and differentiation. WFA increases expression of osteoblast-specific transcription factor and mineralizing genes, promotes osteoblaat survival and suppresses inflammatory cytokines. Data suggests that WFA stimulates bone formation by abrogating proteasomal machinery and provides knowledge base for its clinical evaluation as a bone anabolic agent.

Menopause contributes to bone loss in osteoporosis. Under these conditions, osteoblast-mediated bone formation cannot compensate for accelerated bone resorption, suggesting inhibitory effect on osteoblasts and an increase in adipocytes. The increased bone mass from proteasome inhibition by WFA stimulates osteoblast growth and differentiation, accelerates bone healing following injury and exerts an anabolic effect on osteoporotic bone at about 10 mg/kg/day making it an attractive alternative candidate as a new treatment of post-menopausal osteoporosis.

Aspects of the present disclosure fulfill these needs and provide further related advantages as described in the following summary.

### SUMMARY

Aspects of the present disclosure teach certain benefits in construction and use which give rise to the exemplary advantages described below.

The disclosure relates to the use of topical Withaferin A administered parenterally or topically for a wide range of pharmacological activities, for example for the treatment of cancer.

In one aspect, disclosed herein is a formulation for transdermal delivery of Withaferin A through the skin of a subject, comprising: a therapeutically effective amount of Withaferin A; a buffering agent comprising at least one carbonate salt, lysine, tris, a phosphate buffer and/or 2-imidazole-1-yl-3-ethoxycarbonylpropionic acid (TEPA), or a combination thereof in an amount between about 5-56% w/w; and a penetrant portion in an amount between about 44 to 90% w/w.

In another aspect, disclosed herein is a method for transdermal delivery of a formulation for transdermal delivery of Withaferin A through the skin of a subject, comprising: a therapeutically effective amount of Withaferin A; a buffering agent comprising at least one carbonate salt, lysine, tris, a phosphate buffer and/or 2-imidazole-1-yl-3-ethoxycarbonylpropionic acid (TEPA), or a combination thereof in an amount between about 5-56% w/w; and a penetrant portion in an amount between about 44 to 90% w/w, through the skin of a subject.

Other features and advantages of aspects of the present disclosure will become apparent from the following more detailed description, taken in conjunction with the accompanying drawings, which illustrate, by way of example, the principles of aspects of the disclosure.

### BRIEF DESCRIPTION OF THE DRAWINGS

The accompanying drawings illustrate aspects of the present disclosure. In such drawings:

FIG. 1A illustrates the mode of determining micellar stability in water.

FIG. 1B illustrates the micellar stability in water.

FIG. 2A illustrates the mode of determining micellar stability in acetone.

FIG. 2B illustrates the micellar stability in acetone.

FIG. 3 is a schematic illustration of polymer micelle formation.

FIG. 4 is a schematic of the reverse micellar structures formed by lecithin with and without bile salt.

FIG. 5 shows the effect of adding electrolytes to the bile salt/lecithin micelles resulting in increase in viscosity and stability.

The above described drawing figures illustrate aspects of the disclosure in at least one of its exemplary embodiments, which are further defined in detail in the following description. Features, elements, and aspects of the disclosure that are referenced by the same numerals in different figures represent the same, equivalent, or similar features, elements, or aspects, in accordance with one or more embodiments.

### DETAILED DESCRIPTION

Applicants have found that the drawbacks of intravenous and oral administration of WFA can be overcome by administering these agents non-systemically parenterally, such as by intraperitoneal, subcutaneously or intramuscular injection and alternatively by topical administration. Topical administration is most conveniently transdermal, but further includes transmembrane administration, for example by suppository or intranasal application. The molecular mass of WFA places it well within the range of molecular weight at 470.59772 g/mol to be effectively transported across the epithelial barrier.

Thus, in one aspect, the disclosure is directed to a method to inhibit cancer growth, to induce tumor cell apoptosis, to reinforce cellular antioxidant and/or detoxification, inhibit tumor cell proliferation, to suppress tumor angiogenesis, to block epithelial to mesenchymal transition with tumor invasion and metastasis, to alter tumor cell metabolism, to effect tumor immunomodulation, to eradicate cancer stem cells, and to elicit reinforcement of cancer chemotherapeutic agents.

Other embodiments of the disclosure include the topical administration of WFA for its proteasomic inhibition effects. Among its effects are those that control obesity, exert anti-diabetic effects, and induces its anabolic effects by increasing osteoblastic proliferation and differentiation in the treatment of injury and osteoporosis.

For transdermal topical administration, a suitable formulation typically involves a carrier that enhances penetration of the intact skin and, in some embodiments, is composed of chemical penetration enhancers (CPEs). In some cases, it can also include peptides designed to penetrate cells, i.e., cell penetrating peptides (CPPs), also known as skin penetrating peptides (SPPs), The formulation may be applied as such, or by means of occlusive devices, such as (transdermal) patches.

As noted above, the disclosure is directed to a method and composition to promote the proteasomal inhibitory effects of Withaferin A (WFA), which include a wide range of pharmacological applications. These activities include inhibiting tumor growth through ATP-independent inhibition of heat shock protein 90 in in vivo pancreatic models, the anti-tumor and anti-angiogenesis activity of WFA due to the inhibition of chymotrypsin, reinforcement of cellular antioxidant and/or detoxification systems, suppression of inflammatory pathways, selective inhibition of tumor cell proliferation and induction of apoptosis, suppression of tumor angiogenesis, blockade of epithelial-to-mesenchymal transition, tumor invasion, and metastasis, alteration of tumor cell metabolism, immunomodulation, and eradication of cancer stem cells.

Other embodiments of the disclosure include the topical administration of WFA for its proteasomic inhibition effects. Among its effects are those that control obesity, exert anti-diabetic effects, and induce its anabolic effects by increasing osteoblastic proliferation and differentiation in the treatment of injury and osteoporosis.

Topical administration is most conveniently transdermal, but further includes transmembrane administration, for example by suppository or intranasal application.

Also embodied in this patent is the topical administration of agents and drugs, with or without occlusion in any manner and which are not conjugated with or delivered by means of penetration enhancing formulations but are merely applied to the intact skin with or without massaging the skin for the purpose of breaching the skin's permeation barrier.

The applicant surprisingly discovered that the combination of these two hypothetical mechanisms, functioning in synergy, was successful in TDDD of guest molecules of molecular weights exceeding 500 Da and, in fact, beyond 150 kDa. These two synergistic mechanisms involve different interactions between the SPPs and the cellular moiety in the “transcellular” mechanism and the CPEs in the “extracellular” mechanism.

This disclosure herein demonstrates integrative and cooperative methods with compositions that are directed to the simultaneous and selective disruption of the cellular and lipid matrix contributions to the SC permeation barrier in conjunction with the transdermal delivery of agents. The mode of each physico-chemical component will be presented separately, although they may participate cooperatively in a chemical permeation enhancement (CPE) composition.

While primarily directed to the permeation enhancement of drug delivery for human beings, the application of this disclosure is not limited to humans but has similar application to other members of the animal kingdom.

### The Nature of the Penetrant

While it has been well recognized that the primary efforts employed to enhance SC permeability have focused upon manipulations of the extracellular lipid milieu, little attention has been directed to degrading the cellular components of the SC. This patent embodies an integrative and cooperative transdermal drug delivery formulation that simultaneously disrupts both the extracellular lipid matrix, as well as the cellular contribution to the SC permeation barrier. This patent embodies the application of permeation enhancement formulations directed either to the extracellular lipid matrix, the transcellular structure or both in co-administration. This is to be determined by the nature of the guest cargo, its application, its target site and its molecular weight.

### Cellular Component

The preferred biochemical process, which is directed to the cellular component of the SC permeability barrier, is facilitated by a synergistic action of several biological processes, which combine to enhance transdermal drug delivery. Each of these processes might be used individually.

This patent embodies TD-1, as well as the other cationic cyclo-peptide variants identified as TDR-2, TDR-3 and TDR-7, in which arginine substitutions are made at N-4, N-5 and N-7, and TDK-2, TDK-3 and TDK-7, in which lysine substitutions are made at N-2, N-3 and N-7. Also embodied in this patent is cationic cyclo-peptide variant TD-34 as bis-substitute peptide in N-5 and N-6. The cyclic structure and the disulfide constrained nature is critical for enhancement activity of the peptides. The TDS series of the same amino acid sequence of cyclic structure with TD-1 is further embodied as a modification via substitution of the N-terminal with three amino acids possessing the same cationic group with various side-chain lengths. The enhancement activity has been demonstrated to be proportional to side-chain length and identified as TDS-3>TDS-2>TDS-1.

While the exact mechanism is unclear, our studies have revealed the profound activity of cell penetrating peptides (CPPs) with special reference to TD-1, to be upon interactions with the skin cellular components. The CPPs function by permeating through the transcellular route passing through hydrophilic keratin-packed corneocytes that are embedded in multiple hydrophobic lipid bilayers. While partitioning into the keratin-rich corneocytes, they form bridges that bind with the filamentous keratin α-helices via hydrogen bonds in co-administration as peptide-chaperones without interacting with the guest cargo or degrading the lipid matrix. SPPs, in fact, enhance the lipid organization while simultaneously increasing skin electrical conductivity. TD-1 is non-cytotoxic and non-irritating to skin.

It has been demonstrated that the CPPs also utilize the intercellular pathways via small gaps between the corneocytes by disrupting cell-to-cell junctional desmosomes expeditiously, thereby modifying the normal ultrastructural spacing from about 30 nm to about 466 nm in as little as 30 minutes from topical administration. Transmission electron microscopy has revealed that the intercellular gaps are a transient process that will escort macromolecules across the SC permeation barrier restoring the breaches in about one hour after application.

The co-administration of CPPs has been postulated to result in a statistically significant increase in percentage of α-helices of keratins, suggesting that CPPs stabilize these structural proteins (keratins). The intra-cellular keratins arstabilized by disulfide bonds, which are tightly packed either in α-chains (α-keratins) or in ß-sheet (ß-keratins) structures. The high-degree of cross-linking by the disulfide bonds, hydrophobic interactions and hydrogen bonds between the keratin filament structures within the individual corneocytes confer its mechanical stability preventing free drug transport.

Keratolytic agents will disrupt the tertiary structure and hydrogen bonds between individual keratin filaments, thereby promoting penetration through intact skin. The administration of keratolytic agents will release keratin-bound active drug and enhance bioavailability.

One biochemical process is deployed to disrupt the disulfide linkage of the keratin filaments of which the corneocytes of the SC are comprised. This is contributed by means of a reducing agent containing a thiol moiety. Thioglycolic Acid (TGA) @ 5% concentration is the preferred embodiment. Other agents, such as Dithiothretol (DTT), ß-Mercaptoethanol (ß-ME) and Urea Hydrogen Peroxide @ 17.5% concentration might be similarly employed to act upon the hydrogen bonds, as well as the disulfide bonds.

An additional keratolytic agent or enzyme, such as Proteinase K might be employed to degrade the keratin substrate @ about 10 mg/mL. The optimal pH of keratolytic activity is around pH 8, while activity is detected in a broad range of pH values between 6 to 11 for serine proteases. Chemical hydrolysis will further compromise the barrier property contributed by the corneocytes but the process is irreversible and concentration-dependent.

The simultaneous application of the reducing agent has been demonstrated to have no adverse effect on the keratolytic enzymes and, in fact, allows the preferential access of the enzymes to the substrate for enhanced proteolytic attack.

Sigma-Aldrich offers an appropriate keratinolytic product (K4519-500UN), which is a non-specific serine protease with the capability of degrading insoluble keratin substrates by cleaving non-terminal peptide bonds.

This patent further embodies an alternative to the reducing agent/keratolytic enzyme combination by means of two cooperating enzymes isolated from a keratin-degrading bacterium, Stenotrophomonas sp. strain D-1. These synergistic enzymes disrupt the disulfide bonds while simultaneously degrading the keratin substrate.

### Extra-Cellular Component

Enhancement of transdermal drug delivery directed to the cellular component of the SC barrier is a complex process and, therefore might employ individual CPEs or mixtures of chemicals.

The formulations comprise mixtures wherein the CPEs interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers. (As used herein “detergent” and “surfactant” are synonymous).

The preferred biochemical process, which is directed to the extra-cellular lipid matrix of the SC permeability barrier and is facilitated by the carrier, which preferentially employs additional penetrants described in the cited US2009/0053290 (290) and WO2014/209910 ('910)—i.e., benzyl alcohol and a lecithin organogel, but at much higher ratios of lecithin organogel to benzyl alcohol than in the prior art compositions. The present carriers also may include a nonionic surfactant which is disclosed to be undesirable in the '910 publication and is described in the '290 publication as present only in very low amounts. The applicant has found that by employing very high amounts of the lecithin organogel relative to benzyl alcohol and relative to the weight of the formulation, as well as in some embodiments providing a combination of a nonionic surfactant and molar excess of a polar gelling agent, the penetration capabilities of the resulting formulation and the effective level of delivery of the active agent can be greatly enhanced. Such a result was completely unpredictable as it was believed that relatively equal amounts of the benzyl alcohol and lecithin organogel especially a somewhat higher concentration of benzyl alcohol than lecithin organogel were responsible for the level of penetration achieved by prior art formulations.

Water-in-oil microemulsions have a generic role in the delivery of a wide range of water-soluble molecules from 100 to 150 kDa. The bio-activity is maintained during formulation with microemulsions and during transit through the skin.

Soy lecithin phosphatidylcholine has been revealed to form a noncovalent complex with TD-1, which implies an interaction between TD-1 and the negatively charged cell lipids. Microemulsions consisting of bile salts, lecithin organogel and electrolytes have been used to form supramolecular structure that can increase not only skin permeability but also drug solubility in formulation and drug partitioning into the skin.

Lecithin is a biosurfactant and a zwitterionic phospholipid molecule with a head group having a positively charged choline and a negatively charged phosphate. When a small quantity of water is added to these fluids, the lecithin tends to self-organize into bi-layer membranes and in turn into vesicles or spherical micelles. Water is the most commonly employed polar agent although some other polar agents such as glycerol, ethylene glycol and formamide have been found to possess the capability of transferring an initial non-viscous lecithin solution into a jelly-like state.

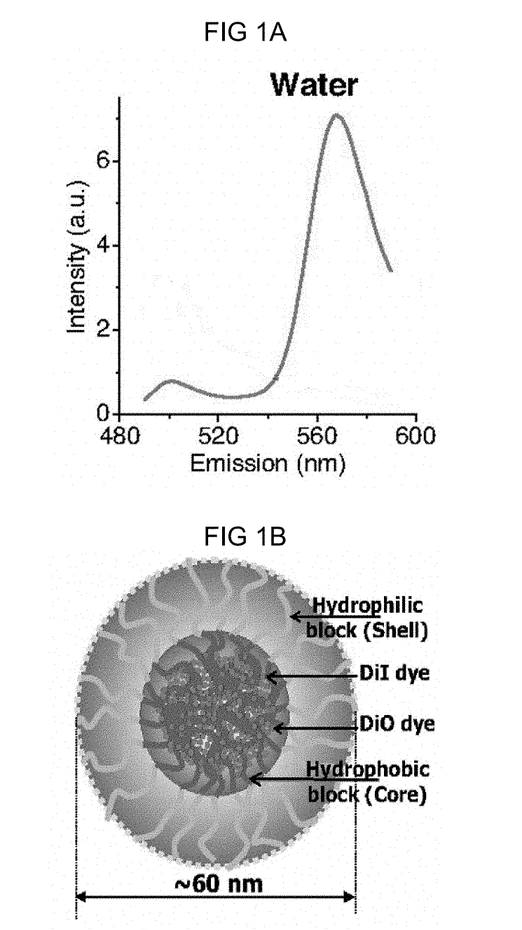

The first examples of such micelles were tertiary mixtures of lecithin-water-oil (organic solvents). While lecithin alone forms vesicles or micelles, these micelles are inherently unstable because the bulky hydrophobic tails of the lipid (lecithin) inhibit its solubility in water (FIG. 1).

The lecithin organogel included in the composition is a combination of lecithin with an organic solvent, which is typically amphiphilic. Suitable organic solvents include, in addition to isopropyl palmitate, ethyl laurate, ethyl myristate and isopropyl myristate. Certain hydrocarbons, such as cyclopentane, cyclooctane, trans-decalin, trans-pinane, n-pentane, n-hexane, n-hexadecane may also be used. The ratio of lecithin to isopropyl palmitate may be 50:50. For examples, a formulation containing soy lecithin in combination with isopropyl palmitate is employed, however, other lecithins could also be used such as egg lecithin or synthetic lecithins. Soy lecithin comprised of 96% pure phosphatidylcholine is preferred.

Various esters of long chain fatty acids may also be included. Methods for making such lecithin organogels are well known in the art. In most embodiments, the lecithin organogel is present in the final formulation in the range of 25-70% w/w and at intermediate percentages such as 30% w/w, 40% w/w, 50% w/w, 60% w/w, etc.

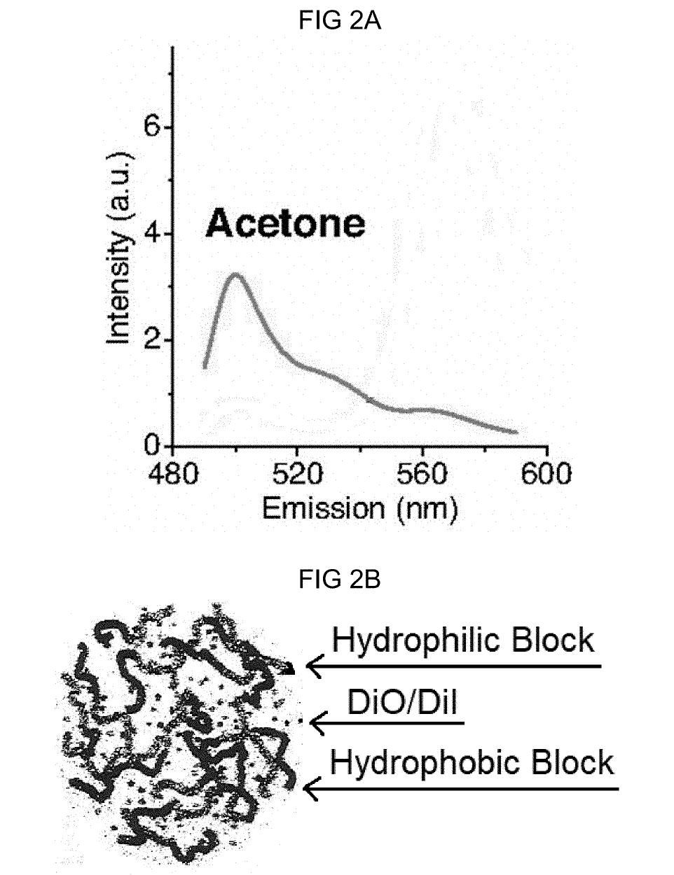

Lecithin organogels may be in the form of vesicles, microemulsions and micellar systems (FIG. 2). In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semisolid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine.

Lecithin microemulsion-based organogels are thermodynamically stable, clear, visco elastic, biocompatible and isotropic phospholipid structured systems. The naturally occurring surfactant, lecithin, can form reverse micelle-based microemulsions in non-polar environment because of its geometric discipline. These small reverse micelles upon addition of a specific amount of water, likely grow mono-dimensionally into long flexible and cylindrical giant micelles, above a critical concentration of lecithin. These giant micelles form a continuous network that immobilizes the external organic phase forming a gel or jelly-like state.

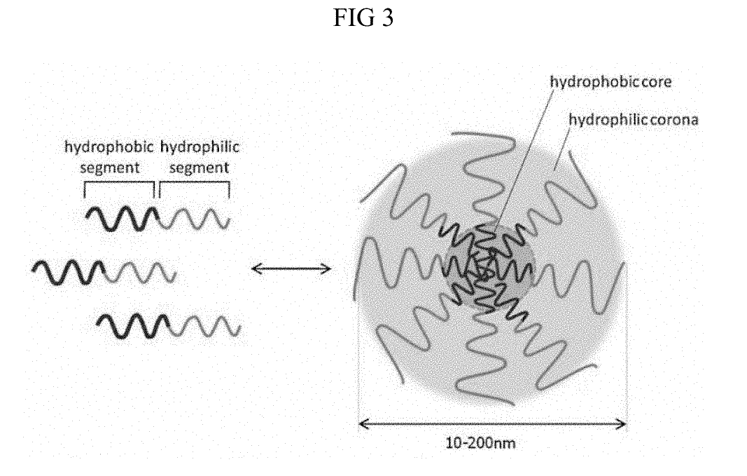

The applicant has further discovered that, while lecithin alone forms vesicles or micelles, these micelles are inherently unstable. The addition of second class of biosurfactants, bile salts, in small amounts will intercalate into lecithin vesicles and stabilize these structures. Accordingly, lecithin-bile salt vesicles have been examined in the context of lipid-protein interactions (FIG. 3).

Alternatively, an anhydrous composition may be obtained by using, instead of a polar component, a material such as a bile salt. When formulated with bile salts, the micellular rheologic nature of the composition is altered so that rather than a more or less spherical vesicular form, the vesicles become wormlike and are able to accommodate larger guest molecules, as well as penetrate the epidermis more effectively.

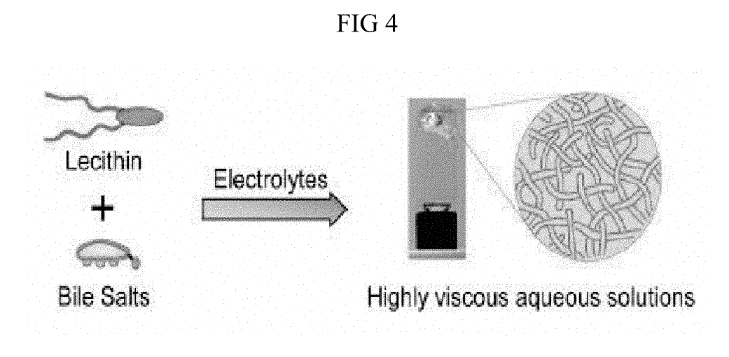

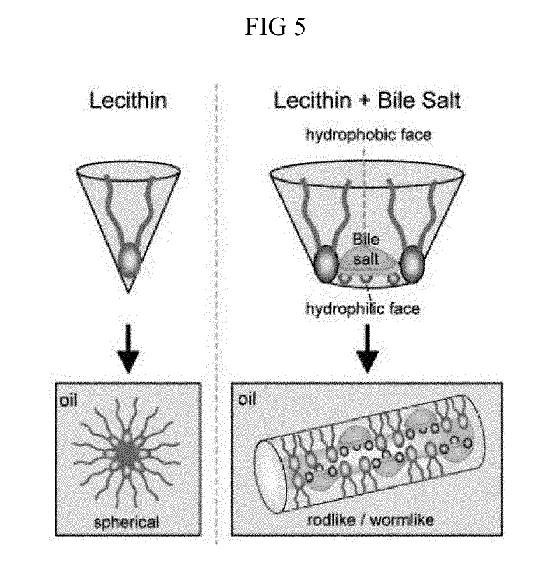

The effective transdermal delivery of drugs is dependent upon three critical factors involved in the self-assembly of micelles; thermodynamic and kinetic stability viscosity and viscoelasticity. Lecithin organogel micelles are inherently unstable, thereby releasing their cargo prematurely before reaching the target site. The introduction of bile salts result in enhanced micellar stability (FIG. 4), viscosity and visco-elasticity. Suitable bile salts include salts of deoxycholic acid, taurocholic acid, glycocholic acid, taurochenodeoxycholic acid, glycochenodeoxycholic acid, cholic acid and the like. Certain detergents, such as Tween® 80 or Span® 80 may be used as alternatives.

The applicant has additionally discovered that the formation of worms also requires a background electrolyte at sufficient levels. These electrolytes, such as sodium citrate, are required to more effectively increase viscosity and visco-elasticity of micelles and screen the repulsion between bile salt anions at a minimal concentration. Another effect of sodium citrate is its ability to “salt out” solutes from water as the Hofmeister effect. In other words, a specific molar ratio and a sufficient electrolyte concentration are required for the formation of stable, long flexible cylindrical micelles.

The percentage of these components in the anhydrous forms of the composition is in the range of 1% w/w-15% w/w. In some embodiments, the range of bile salt content is 2%-6% w/w or 1%-3.5% w/w. In these essentially anhydrous forms, powdered or micronized nonionic detergent is used to top off, typically in amounts of 20%-60% w/w. In one approach to determine the amount of bile salt, the % is calculated by dividing the % w/w of lecithin by 10.

It is now widely recognized that these bile salt-stabilizing vesicles are very similar to polymeric chains with the important exception that these vesicles are in thermal equilibrium with their monomers and break and recombine at a rapid rate. A competition between vesicular breaking and chain reptation dictates the rheology of the fluid. Recent studies have focused on the role played by the water in reverse micellar growth (water can be substituted with other polar solvents, such as glycerol). These studies have yielded disparate and sometimes diverging conclusions; some have speculated that water is the necessary glue that holds these reverse micelles together, but this has been refuted by others. These spherical vesicles grow axially into flexible cylinders. Thus, the crucial component is water and the molar ratio of water to lecithin (denoted by w₀) is the key parameter in dictating micellar growth.

In embodiments where a bile salt is added to the combination of benzyl alcohol and lecithin organogel in lieu of topping off with an aqueous medium, micelles that would have been relatively spherical may become elongated and worm-like thus permitting superior penetration of the stratum corneum of the epidermis. The worm like formation of the micelles is particularly helpful in accommodating higher molecular weight therapeutic agents. Bile salts are facial amphiphiles and include salts of taurocholic acid, glycocholic acid, taurochenodeoxycholic acid, glycochenodeoxycholic acid, cholic acid, deoxycholic acid. Other detergents are also useful in lieu of bile salts, and include Tween® 80 and Span® 80.

The inclusion of these bile salts facilitates the ultradeformability of micelles which, in turn, facilitate passage of low and high molecular weight drugs and other active agents, such as nucleic acids and proteins. These compositions overcome the skin penetration barrier by squeezing themselves along the intercellular sealing lipid thereby following the natural gradient across the stratum corneum. This facilitates a change in membrane composition locally and reversibly when pressed against or attracted to a narrow pore.

Bile salts in combination with lecithin organogel facilitate the factors of micellar stability, enhanced viscosity and visco-elasticity so critical in transdermal drug delivery. Both thermodynamic and kinetic stability is enhanced by the addition of background electrolytes, such as sodium chloride and sodium citrate. Sodium citrate is the more effective electrolyte because it is strongly ionic, thereby reinforcing the interactions between water molecules and various solutes. These electrolytes can more effectively increase viscosity and visco-elasticity of micelles and screen the repulsion between bile salt anions at a minimal concentration.

The molar ratio of bile salt to lecithin is 1:1, but the concentration of electrolyte is determined by titration of the solution to transparency of the solution and enhanced viscosity as determined when the solution container is inverted.

In some formulations of the disclosure, in addition to the above amounts of bile salts, benzyl alcohol, lecithin organogel and active ingredient, the formulations are “topped off” with a powdered nonionic detergent. The pH of such compositions can be determined by taking a small sample and dissolving it in water to test the appropriate pH. In many embodiments, the pH is in the range of 8.5-11 or 9-11 or 10-11.

An additional required component in the formulations of the disclosure is an alcohol. Benzyl alcohol in some formulations but other alcohols could be included, in particular derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are included.

Due to the aromatic group present in a permeation enhancement formulation such as benzyl alcohol, the molecule has a polar end (the alcohol end) and a non-polar end (the benzene end). This enables the agent to dissolve a wider variety of drugs and agents. The alcohol concentration is substantially lower than the concentration of the lecithin organogel in the composition.

By formulating active ingredients in the presence of at least a combination of a lecithin organogel and a suitable alcohol, especially benzyl alcohol where the lecithin organogel is in a ratio of concentration at least 10-fold that of the alcohol on a weight basis, superior results are achieved as illustrated in the examples below.

In some embodiments, as noted above, the performance of the formulations is further improved by including a nonionic detergent and polar gelling agent or including bile salts and a powdered surfactant. In both aqueous and anhydrous forms of the composition, detergents, typically nonionic detergents are added. In general, the nonionic detergent should be present in an amount of at least 2% w/w to 60% w/w. Typically, in the compositions wherein the formulation is topped off with a polar or aqueous solution containing detergent, the amount of detergent is relatively low—e.g., 2%-25% w/w, or 5-15% w/w or 7-12% w/w.

However, in compositions comprising bile salts that are essentially anhydrous and are topped-off by powdered detergent, relatively higher percentages are usually used—e.g., 20%-60% w/w. The boundaries are not rigid but the above description indicates the general range.

In some embodiments, the nonionic detergent provides suitable handling properties whereby the formulations are gel-like or creams at room temperature. To exert this effect, the detergent, typically a poloxamer, is present at a level of at least 9% w/w, preferably at least 12% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohol and copolymers of hydrophilic and hydrophobic monomers where blocks of hydrophilic and hydrophobic portions are used.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by SigmaAldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like.

In the presence of a polar gelling agent, such as water, glycerol, ethylene glycol or formamide, a micellar structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition. As noted above, it may be rather than a polar gelling agent, a bile salt can be used. In this case, the detergent is added in solid, powdered form.

The percentage of active agent in the formulation will depend upon the concentration required to be delivered in order to have a useful effect on treating the disorder. In general, the active ingredient may be present in the formulation in an amount as low as 0.01% w/w up to about 50% w/w. Typical concentrations include 0.25% w/w, 1% w/w, 5% w/w, 10% w/w, 20% w/w and 30% w/w. Since the required percentage of active ingredient is highly variable depending on the active agent and depending on the frequency of administration, as well as the time allotted for administration for each application, the level of active ingredient may be varied over a wide range and is limited only by the necessity for including in the formulation aids in penetration of the skin by the active ingredient.

The formulations of the disclosure may include only one active agent or a combination of active agents. In the present application, “active agent” or “active ingredient” refers to a compound or drug that is active against the factors or agents that result in the desired therapeutic or other localized systemic effect.

In general, in the present application, “a,” “an,” “one,” and the like should be interpreted to mean one or more than one unless it is clear from the context that only a single referent is intended. Thus, “an active ingredient” may refer to one or more such active ingredients.

The formulations of the disclosure may be prepared in a number of ways. Typically, the components of the formulation are simply mixed together in the required amounts. However, it is also desirable in some instances to, for example, carry out dissolution of an active ingredient and then add a separate preparation containing the components aiding the delivery of the active ingredients in the form of a carrier. The concentrations of these components in the carrier, then, will be somewhat higher than the concentrations required in the final formulation

Alternatively, some subset of these components can first be mixed and then “topped off” with the remaining components either simultaneously or sequentially. The precise manner of preparing the formulation will depend on the choice of active ingredients and the percentages of the remaining components that are desirable with respect to that active ingredient.

As noted above, the essential components of the formulations for most applications are 25%-70% w/w lecithin organogel and 0.5-20% w/w benzyl alcohol or closely related alcohol as well as supplementary components such as detergents, typically nonionic detergents, bile salts, polar solvents and the like.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature otherwise stated. The concentration of hydroxypropylcellulose may range from about 1% w/w to about 2% w/w of the composition. Other gelling agents are known in the art and can be used in place of, or in addition to, hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Typical components that may also be included in the formulations are fatty acids, terpenes, lipids, and cationic and anionic detergents.

Other solvents and related compounds that may be used in some embodiments include acetamide and derivatives, acetone, n-alkanes (chain length between 7 and 16), alkanols, diols, short-chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefossé, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C3-C6 triols or their mixtures and a polar lipid compound selected from C16 or C18 monounsaturated alcohol, C16 or C18 branched saturated alcohol and their mixtures, propylene glycol, sorbitan monolaurate sold as Span® 20 sold by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol.

Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N,N-dimethylamino acetate.

Examples of suitable fatty acid include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid.

Examples of suitable fatty alcohol ethers include α-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′dihydroxypropyl)glycerol).

Examples of completing agents that may be used in some embodiments include β- and γ-cyclodextrin complexes, hydroxypropyl methylcellulose (such as Carbopol® 934), liposomes, naphthalene diamide diimide, and naphthalene diester diimide.

One or more anti-oxidants may be included, such as vitamin C, vitamin E, proanthocyanidin and α-lipoic acid typically in concentrations of 0.1%-2.5% w/w.

In some applications, it is desirable to adjust the pH of the formulation to assist in permeation or to adjust the nature of the active agent and/or of the target compounds in the subject. In some instances, the pH is adjusted to a level of pH 9-11 or 10-11 which can be done by providing appropriate buffers or simply adjusting the pH with base.

Skin's electrical resistance or impedance is generally considered a marker of skin permeability and changes in skin resistance due to exposure to different CPEs has been shown to correlate with increased skin permeability to model drug compounds. From a mechanistic viewpoint, skin's electrical resistance is known to be governed primarily to the highest ordered, lipophilic barrier of the SC lipid bilayers. Therefore, changes in skin's resistance are a sensitive measure of changes in the SC lipid bilayer integrity. Changes in skin's resistance are seen to occur with a lag time of one or more hours, which suggests a kinetic barrier that may be a diffusive transport limitation.

Measurement of skin's resistance or impedance can be used to as a ‘generic’ measurement of skin permeability that does not depend on the specific characteristics of target molecules, such as hydrophobicity and charge.

The electrical resistance or impedance across the epidermis was measured as the CPE was applied to the skin, which was maintained at 32° C. Electrical conductivity was calculated from electrical resistance measurements.

In some formulations, formation of micelles is enhanced by milling. The level of enhancement is determined by the pressure and speed at which milling occurs as well as the number of passes through the milling machine. As the number of passes and the pressure is increased, the level of micelle formulation is enhanced as well. In general, increasing the pressure and increasing the speed of milling enhances the level of micelle density.

When the ointment milling machine is a Dermamill 100 (Blaubrite) marketed by Medisca®, typical speeds include any variation between 1 to 100, where 1 is the slowest speed and 100 is the fastest speed, such as speeds of 1, 5, 10, 15, 20, 25, 30, 35, 40, 45, 50, 55, 60, 65, 70, 75, 80, 85, 90 or 100, or any speed in between. The pressure is selected from 1 to 5, where 1 is the highest pressure and 5 is the lowest pressure. The pressure used can be selected from 1, 2, 3, 4, or 5. The number of passes can also be varied, where a pass is complete when all of the product has passed through the rollers of the machine. Multiple passes, such as 2, 3, 4, 5, 6, 7, 8, 9, 10 or more passes, are contemplated in some embodiments. The speed and pressure can be varied for each pass. For example, a first pass may have a first pressure and first speed, while a second (or subsequent) pass may have a second pressure and second speed, where the second pressure is the same or different from the first pressure and the second speed is the same or different from the first speed. The desired micelle density for particular formulations can be determined empirically by varying the speed, pressure and number of passes.

Of course, alternative ointment milling machines could also be used, and comparable speeds, pressures and numbers of passes are replicated by comparison to the equivalents on the Dermamill 100. Alternatively, micelle densities can be compared microscopically to assure equivalent results to those set forth herein. In some embodiments, the micelle density is at least 20%, and in many cases at least 30%, 50%, 70%, 80% or 90% and all levels within this range.

These studies have yielded specific and rare binary mixtures of CPEs in synergistic combination that enhance skin permeability to hydrophilic macromolecules by more than 50-fold without inducing skin irritation.

The preferred embodiment of this patent is an integrative cooperative formulation combining: (1) binary mixtures of CPEs selected from electrometric screening, (2) dual biosurfactant-based reverse wormlike-micellar systems, (3) bipolar aliphatic alcoholic solvents, (4) keratinolytic agents, (5) thiol-moiety reducing agents, and (6) skin penetrating peptides (SPPs) in a higher ordered topical transdermal drug delivery composition, which effectively hosts various guest drug molecules, thereby, breaching the SC permeation barrier and rendering them bioavailable to their target site.

In one aspect, disclosed herein is a formulation for transdermal delivery of Withaferin A through the skin of a subject, comprising: a therapeutically effective amount of Withaferin A; a buffering agent comprising at least one carbonate salt, lysine, tris, a phosphate buffer and/or 2-imidazole-1-yl-3-ethoxycarbonylpropionic acid (TEPA), or a combination thereof in an amount between about 10-56% w/w; and a penetrant portion in an amount between about 44 to 90% w/w.

In some embodiments, the penetrant portion comprises water in an amount less than about 85% w/w.

In some embodiments, the formulation comprises less than about 12% w/w lecithin.

In some embodiments, the penetrant portion further comprises a detergent portion in an amount between about 1 to 70% w/w.

In some embodiments, the buffering agent is in an amount between about 7.5-36% w/w of the formulation.

In some embodiments, the penetrant portion is in an amount between about 44-80% w/w of the formulation.

In some embodiments, the water is in an amount between about 15-42% w/w of the penetrant portion of the formulation.

In some embodiments, the penetrant portion comprises an alcohol in an amount less than 10% w/w of the formulation.

In some embodiments, the penetrant portion comprises lecithin organogel, an alcohol, a surfactant, and a polar solvent.

In some embodiments, the penetrant portion comprises a mixture of xanthan gum, lecithin, sclerotium gum, pullulan, or a combination thereof in an amount less than 5% w/w of the formulation.

In some embodiments, the penetrant portion comprises a mixture of caprylic triglycerides and capric triglycerides in amount less than 8% w/w of the formulation.

In some embodiments, the penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 12% w/w of the formulation.

In some embodiments, the penetrant portion comprises cetyl alcohol in amount less than 5% w/w of the formulation.

In some embodiments, the penetrant portion comprises stearic acid in an amount less than 5% w/w of the formulation.

In some embodiments, the formulation comprises a gelling agent in an amount less than 5% w/w of the formulation.

In some embodiments, the detergent portion comprises a nonionic surfactant in an amount between about 2-25% w/w of the penetrant portion; and a polar solvent in an amount less than 5% w/w of the penetrant portion.

In some embodiments, the carbonate salt is sodium bicarbonate milled to a particle size less than 70 μm, wherein the sodium bicarbonate is solubilized in the formulation in an amount less than 10% w/w of the formulation.

In some embodiments, wherein the formulation further comprises tranexamic acid in an amount less than 5% w/w of the formulation.

In some embodiments, wherein the formulation further comprises a polar solvent in an amount less than 5% w/w of the formulation.

In some embodiments, wherein the formulation further comprises a humectant, an emulsifier, an emollient, or a combination thereof.

In some embodiments, wherein the formulation further comprises the formulation has a pH of 7-10.5.

In another aspect, disclosed herein is a method for transdermal delivery of the formulation of claim 1, through the skin of a subject.

In another aspect, disclosed herein is a method to inhibit cancer growth, to induce tumor cell apoptosis, to reinforce cellular antioxidant and/or detoxification, inhibit tumor cell proliferation, to suppress tumor angiogenesis, to block epithelial to mesenchymal transition with tumor invasion and metastasis, to alter tumor cell metabolism, to effect tumor immunomodulation, to eradicate cancer stem cells, and to elicit reinforcement of cancer chemotherapeutic agents.

Other embodiments of the disclosure include the topical administration of WFA for its proteasomic inhibition effects. Among its effects are those that control obesity, exert anti-diabetic effects, and induces its anabolic effects by increasing osteoblastic proliferation and differentiation in the treatment of injury and osteoporosis.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof. Ultimately, the formulations may be utilized in virtually any context where buffering therapy with or without a therapeutic agent(s) is desired.

### Example 1: Integrative Cooperative CPE Formulation Directed to the Extra-Cellular Matrix to which Might be Added Selected Cysteine Cathepsin Protease-Inhibitors, with or without a Suitable Buffering Agent

1. Cetyltrimethyl ammonium bromide (from about 2.0% to about 10.0%)

2. Sodium cholate: Lecithin (96% pure): Isopropyl myristate (equi-molar 1:1:1 (from about 10% to about 40.0%)

3. Sodium citrate (titrate to transparency/incr. viscosity of #2.)

4. Benzyl alcohol (from about 2.0% to about 30.0%)

5. Cis-Palmitoleic acid (from about 20.0% to about 30% of BA)

6. Methyl pyrrolidone (0.4%)/Dodecyl pyridinium (1.1%) (from about 0.5% to about 5.0%)

7. Pluronic 127 (qs to 100%)

### Example 2: Formulation Directed to the Cellular Component of the SC Permeability Barrier to which Might be Added Selected Cysteine Cathepsin Protease-Inhibitors, with or without a Suitable Buffering Agent

1. ACSSSPSKHCG, [alanine-cysteine-serine-serine-serine-proline-serine-lysine-hisitidine-cysteine-glycine] identified as TD-1

2. Thioglycolic Acid (TGA) (from about 2.0% to about 7.0% concentration) [may be substituted by other reducing agents]

3. Proteinase K (from about 5 mg/mL to about 15 mg/mL)

In closing, regarding the exemplary embodiments of the present disclosure as shown and described herein, it will be appreciated that the formulations disclosed herein are configured for buffering therapy with or without an additional therapeutic agent. Because the principles of the disclosure may be practiced in a number of configurations beyond those shown and described, it is to be understood that the disclosure is not in any way limited by the exemplary embodiments but is generally directed to a transdermal formulation and is able to take numerous forms to do so without departing from the spirit and scope of the disclosure. It will also be appreciated by those skilled in the art that the present disclosure is not limited to the particular components disclosed but may instead entail other functionally comparable formulation components, now known or later developed, without departing from the spirit and scope of the disclosure.

Certain embodiments of the present disclosure are described herein, including the best mode known to the inventor(s) for carrying out the disclosure. Of course, variations on these described embodiments will become apparent to those of ordinary skill in the art upon reading the foregoing description. The inventor(s) expect skilled artisans to employ such variations as appropriate, and the inventor(s) intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present disclosure (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the disclosure otherwise claimed. No language in the present specification should be construed as indicating any non-claimed element essential to the practice of the disclosure.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

While aspects of the disclosure have been described with reference to at least one exemplary embodiment, it is to be clearly understood by those skilled in the art that the disclosure is not limited thereto. Rather, the scope of the disclosure is to be interpreted only in conjunction with the appended claims and it is made clear, here, that the inventor(s) believe that the claimed subject matter is the disclosure.

What is claimed is:

1. A formulation for transdermal delivery of Withaferin A through the skin of a subject, comprising:
a therapeutically effective amount of Withaferin A;
a buffering agent comprising at least one carbonate salt, lysine, tris, a phosphate buffer and/or 2-imidazole-1-yl-3-ethoxycarbonylpropionic acid (IEPA), or a combination thereof in an amount between about 5-56% w/w; and
a penetrant portion in an amount between about 44 to 95% w/w.

2. The formulation of claim 1, wherein the penetrant portion comprises water in an amount less than about 85% w/w.

3. The formulation of claim 1, wherein the formulation comprises less than about 12% w/w lecithin.

4. The formulation of claim 1, wherein the penetrant portion further comprises a detergent portion in an amount between about 1 to 70% w/w.

5. The formulation of claim 1, wherein the buffering agent is in an amount between about 7.5-36% w/w of the formulation.

6. The formulation of claim 1, wherein the penetrant portion is in an amount between about 44-80% w/w of the formulation.

7. The formulation of claim 1, wherein the water is in an amount between about 15-42% w/w of the penetrant portion of the formulation.

8. The formulation of claim 1, wherein the penetrant portion comprises an alcohol in an amount less than 10% w/w of the formulation.

9. The formulation of claim 7, wherein the penetrant portion comprises lecithin organogel, an alcohol, a surfactant, and a polar solvent.

10. The formulation of claim 1, wherein the penetrant portion comprises a mixture of xanthan gum, lecithin, sclerotium gum, pullulan, or a combination thereof in an amount less than 5% w/w of the formulation.

11. The formulation of claim 1, wherein the penetrant portion comprises a mixture of caprylic triglycerides and capric triglycerides in amount less than 8% w/w of the formulation.

12. The formulation of claim 1, wherein the penetrant portion comprises lecithin, phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 12% w/w of the formulation.

13. The formulation of claim 1, wherein the penetrant portion comprises cetyl alcohol in amount less than 5% w/w of the formulation.

14. The formulation of claim 1 wherein the penetrant portion comprises stearic acid in an amount less than 5% w/w of the formulation.

15. The formulation of claim 1, wherein the formulation comprises a gelling agent in an amount less than 5% w/w of the formulation.

16. The formulation of claim 2, wherein the detergent portion comprises a nonionic surfactant in an amount between about 2-25% w/w of the penetrant portion; and a polar solvent in an amount less than 5% w/w of the penetrant portion.

17. The formulation of claim 1, wherein the carbonate salt is sodium bicarbonate milled to a particle size less than 70 μm, wherein the sodium bicarbonate is solubilized in the formulation in an amount less than 10% w/w of the formulation.

18. The formulation of claim 1, further comprising tranexamic acid in an amount less than 5% w/w of the formulation.

19. The formulation of claim 1, further comprising a polar solvent in an amount less than 5% w/w of the formulation.

20. The formulation of claim 1, further comprises a humectant, an emulsifier, an emollient, or a combination thereof.

21. The formulation of claim 1, wherein the formulation has a pH of 7-10.5.

22. A method for transdermal delivery of the formulation of claim 1, through the skin of a subject.
